(19) United States
(12) Patent Application Publication
(10) Pub. No.: US 2002/0151057 A1
(43) Pub. Date: Oct. 17, 2002

(54) Methods for generating doubled haploid maize plants

(76) Inventors: Yuanming Zheng, Pullman, WA (US); Calvin F. Konzak, Pullman, WA (US); Yujia Weng, Pullman, WA (US); Sahibzada Rafiullah, Pullman, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 10/037,448

(22) Filed: Jan. 2, 2002

Related U.S. Application Data
(63) Continuation-in-part of application No. 09/383,588, filed on Aug. 26, 1999, now Pat. No. 6,362,393.
(60) Provisional application No. 60/260,028, filed on Jan. 5, 2001.

(51) Int. Cl. C12N 5/00; C12N 5/02

(52) U.S. Cl. 435/424; 435/430

(57) ABSTRACT

In one aspect, the present invention provides methods for generating doubled haploid and/or haploid maize plants from microspores. The methods of this aspect of the invention include the steps of: (a) selecting maize plant material comprising maize microspores at a developmental stage amenable to androgenic induction; (b) incubating the microspores in incubation medium at a temperature and osmolarity effective to induce androgenesis to obtain temperature-treated microspores; (c) isolating the temperature-treated microspores; (d) cultivating the isolated, temperature-treated microspores in cultivation medium with either at least one live plant ovary and/or ovary-conditioned medium to produce regenerative maize tissue, wherein the cultivation medium has an osmolarity between about 300 mOsm and about 500 mOsm and comprises at least one cytokinin and at least one auxin; and (e) regenerating maize plants from the regenerative maize tissue. The present invention also provides methods for producing regenerative maize tissue from maize microspores.

### CROSS-REFERENCES TO RELATED APPLICATIONS

This application claims the benefit of priority to U.S. Provisional Application No. 60/260,028, filed on Jan. 5, 2001, under U.S.C. § 119, and is a continuation-in-part of U.S. application Ser. No. 09/383,588, filed Aug. 26, 1999, to which benefit of priority is claimed under 35 U.S.C. 120.

### FIELD OF THE INVENTION

This invention relates to methods for generating doubled haploid maize plants from microspores, and to doubled haploid maize plants produced by the methods disclosed herein.

### BACKGROUND OF THE INVENTION

Maize is the third most important crop after wheat and rice (FAO Yearbook Production, Vol. 47, 1997, FAO Statistics Series No. 117). It is estimated that maize is produced on nearly 130 million hectares over many countries of the world with a worldwide production of 500,000 metric tons. Maize production is limited mostly by growing season length and temperature, but breeders have continued to develop new varieties that expand the production area. The crop is widely grown and serves as food for direct human consumption as well as for animal feed. Since the early 20th century, maize yield has substantially increased due largely to the use of hybrids. Breeding hybrids normally requires the development of inbred lines by repeated self-pollination. Six to eight generations of self-pollination are required following heterozygous crosses to achieve sufficient homozygosity for an inbred line. During this period, selection may be made for agronomic characters and combining ability. Another three to four generations are required for field testing of hybrid crosses. Together, about six to eight years of development are needed to develop inbreds for new maize hybrids.

One possible way to reduce the time required for hybrid development is to produce them from the gametic cells as haploid plants, the chromosomes of which may double spontaneously or can be doubled using colchicine or other means to achieve homozygous, doubled-haploid plants. In particular, doubled haploids can be produced from the microspores which normally give rise to pollen grains.

The life cycle of flowering plants exhibits an alteration of generations between a sporophytic (diploid) phase and a gametophytic (haploid) phase. Meiosis produces the first cells of the haploid generation which are either microspores (male) or megaspores (female). Microspores divide and develop within anthers to become mature male gametophytes (pollen). By producing doubled-haploid progeny, the number of possible gene combinations for any number of inherited traits is more manageable. Thus, marked improvements in the economics of breeding can be achieved via doubled haploid production, since selection and other procedural efficiencies can be markedly improved by using true-breeding (homozygous) progenies. With doubled haploid production systems, homozygosity is achieved in one generation. Thus, the breeder can eliminate the numerous cycles of inbreeding necessary by conventional methods to achieve practical levels of homozygosity. Indeed, true homozygosity for all traits is not even achievable by conventional breeding methods.

Consequently, a doubled haploid technology enables maize breeders to reduce the time and the cost of inbred and hybrid plant development relative to conventional breeding practices. Thus, there is a need for methods of producing doubled haploid plants that are applicable to maize.

### SUMMARY OF THE INVENTION

In accordance with the foregoing, in one aspect the present invention provides methods of generating doubled haploid and/or haploid plants from maize microspores.

The methods of the present invention for producing maize plants from maize microspores include the steps of: selecting plant material including microspores at a developmental stage amenable to androgenic induction; incubating the microspores in incubation medium at a temperature and osmolarity effective to induce androgenesis; isolating the temperature-treated microspores; cultivating the isolated, temperature-treated microspores in cultivation medium containing at least one cytokinin and at least one auxin, and having an osmolarity between about 300 mOsm and about 500 mOsm, with at least one live plant ovary and/or ovary-conditioned medium to produce regenerative maize tissue; and regenerating maize plants from the regenerative maize tissue.

In the practice of this aspect of the present invention, plant material is selected that bears reproductive organs containing microspores at a developmental stage that is amenable to androgenic induction. In preferred embodiments, the microspores are in the late-uninucleate to early-binucleate stage of development. In some embodiments, the selected plant material is tassels bearing florets. The microspores are incubated in incubation medium under temperature conditions effective to induce androgenesis. In some embodiments, microspores are incubated at a temperature from about 1° C. above the freezing point of the incubation medium to about 17° C. In some embodiments, microspores are incubated at a temperature between about 4° C. and about 10° C. In some embodiments, microspores are incubated at a temperature between about 8° C. and about 10° C. In some embodiments of the methods of this aspect of the invention, the duration of the temperature treatment is from about 3 to about 21 days, such as from about 7 days to about 15 days, or from about 10 days to about 15 days.

The osmolarity of the incubation medium is typically from about 300 mOsm to about 450 mOsm. Some embodiments of the incubation medium includes an effective amount of at least one sporophytic development inducer, such as 2-hydroxynicotinic acid (2-HNA), which switches microspores from gametophytic to sporophytic development. The microspores may optionally be subjected to nutrient stress, for example during the temperature treatment.

The temperature-treated microspores can be isolated by any suitable means, such as by grinding the treated plant tissue with a mortar and pestle, filtering the ground plant tissue, and separating viable temperature-treated microspores from other plant material, for example by subjecting the filtrate to density centrifugation.

The isolated, temperature-treated microspores are cultivated under controlled osmolarity conditions in a cultivation medium with least one live plant ovary and/or ovary-conditioned medium to produce regenerative maize tissue. In some embodiments, the cultivation medium is refreshed periodically to maintain the osmolarity of the medium between about 300 mOsm and about 500 mOsm. In some embodiments, the cultivation medium includes a combination of sucrose and maltose as the carbon source.

In some embodiments, the cultivation medium includes an effective amount of auxin. In some embodiments, the auxins used in the cultivation medium are 2,4-dichlorophenoxyacetic acid (2,4-D) and phenylacetic acid (PAA). In some embodiments, the cultivation medium includes an effective amount of at least one cytokinin, for example kinetin.

The regenerative maize tissue is regenerated into mature maize plants. In some embodiments, regenerative maize tissue is transferred to a shoot regeneration medium. Some embodiments of the shoot regeneration medium include an effective amount of cytokinins, preferably kinetin and benzaminopurine (BAP). In some embodiments, the shoot regeneration medium comprises an effective amount of an auxin, such as naphthalene acetic acid (NAA).

In some embodiments, regenerative maize tissue with shoots is transferred to a root regeneration medium. In some embodiments, the composition of the root regeneration medium is identical to the composition of the shoot regeneration medium, but without auxins or cytokinins. Regenerative tissue of poor quality can be transferred to a competency medium before transferring the regenerative maize tissue to shoot regeneration medium. In some embodiments, the competency medium is supplemented with an auxin, such as 2,4-D.

Optionally, the microspores can be contacted with a cell spindle inhibiting agent, such as pronamide, or a gibberellin before, during, after, or overlapping with any portion of the temperature treatment.

The resulting plants may be doubled haploids, or they may be haploids which can be converted to doubled haploids by treatment with a chromosome doubling agent such as colchicine (see, e.g., U.S. Pat. No. 5,445,961, which is incorporated herein in its entirety).

In another aspect of the invention, methods are provided for producing regenerative maize tissue from maize microspores. The methods include the steps of:

selecting plant material including microspores at a developmental stage amenable to androgenic induction; incubating the microspores in incubation medium at a temperature and osmolarity effective to induce androgenesis; isolating the temperature-treated microspores; and cultivating the isolated, temperature-treated microspores in cultivation medium containing at least one cytokinin and at least one auxin, and having an osmolarity between about 300 mOsm and about 500 mOsm, with at least one live plant ovary and/or ovary-conditioned medium to produce regenerative maize tissue.

The methods of the present invention for producing regenerative maize tissue or mature maize plants from maize microspores may optionally include the step of genetically transforming the microspores. Microspores can be genetically transformed at any time during treatment of the microspores in accordance with the methods of the present invention. Thus, in one aspect, the present invention provides genetically transformed maize plants regenerated from microspores.

In another aspect of the present invention, doubled haploid and/or haploid plants are provided that are produced according to the methods of the present invention.

The methods of the present invention are useful for producing mature maize plants, or regenerative maize tissue, from maize microspores. The methods of the present invention can be used, for example, to produce numerous genetically identical maize plants from microspores obtained from a single maize plant possessing one or more desirable characteristics. The methods of the present invention can therefore be incorporated into a maize breeding program to produce populations of maize plants possessing one or more desirable characteristics.

### DETAILED DESCRIPTION OF THE PREFERRED EMBODIMENT

Unless specifically defined herein, all terms used herein have the same meaning as they would to one of ordinary skill in the art.

The term “doubled haploid” is used herein to refer to plants produced by doubling the chromosome number of a gamete-derived haploid plant which is produced via male gamete sporophytic divisions. The chromosome doubling (including spontaneous chromosome doubling) can occur at any stage in the process of converting a microspore to a whole plant, or can be induced, for example, by treating haploid plants with colchicines, or other cell spindle inhibitors.

The term “androgenic induction” means induction of androgenesis, i.e., the process by which microspores produce calli and embryoids, which can regenerate into plants. The term “microspore” refers herein to the male gametophyte of a plant, including all stages of development from meiosis through formation of the mature pollen grain.

The term “auxins” as used herein refers to naturally occurring auxins, auxin precursors, and synthetic analogues of auxins and auxin precursors. An example of a naturally occurring auxin is indole acetic acid (IAA). An example of a synthetic IAA-like auxin is 2,4-dichlorophenoxyacetic acid (2,4-D). An example of an auxin precursor is phenylacetic acid (PAA).

The term “regenerative maize tissue” as used herein refers to any tissue derived from microspores treated in accordance with the methods of the invention that has the potential to yield mature maize plants when treated in accordance with the methods of the invention. Thus, the term “regenerative maize tissue” includes embryoids, pro-embryoids, and calli. The term “embryoid” refers to an embryo-like, multi-cellular structure that is derived from in vitro culture and that can develop into a plant. The term “pro-embryoid” refers to a smaller, immature stage of an embryoid that exhibits some of the morphological features of an embryoid. The term “callus” refers to a mass of undifferentiated cells that do not usually exhibit polarity.

The abbreviation mg/l means milligrams per liter.

The abbreviation mOsm means milliOsmoles. The osmotic concentration, or osmolarity, is expressed in units of milliOsmoles per kg of water, where one milliOsmole is equivalent to one millimole of dissolved solute particles.

In one aspect, the present invention provides methods of generating doubled haploid and/or haploid maize plants from maize microspores. The methods of the present invention for producing maize plants from maize microspores include the steps of: selecting plant material including microspores at a developmental stage amenable to androgenic induction; incubating the microspores in incubation medium at a temperature and osmolarity effective to induce androgenesis; isolating the temperature-treated microspores; cultivating the isolated, temperature-treated microspores in cultivation medium containing at least one cytokinin and at least one auxin, and having an osmolarity between about 300 mOsm and about 500 mOsm, with at least one live plant ovary and/or ovary-conditioned medium to produce regenerative maize tissue; and regenerating maize plants from the regenerative maize tissue.

In the practice of this aspect of the present invention, plant material is selected that includes microspores at a developmental stage amenable to androgenic induction. For example, the selected plant material can be all of the maize inflorescence, or any part thereof that contains microspores. In some embodiments, the selected plant material is tassels bearing florets. In some embodiments, the selected plant material is anthers. In some embodiments, the selected plant material includes any part of the inflorescence that contains microspores.

In one embodiment, fresh maize tassels are cut at 1-2 nodes below the tassel base. Leaves are trimmed from the selected tassels, and the tassels are placed in a flask, preferably an Ehrlenmeyer flask, containing sterile, distilled water. Several tassels may be placed in the same flask. The flask containing the tassels may be placed inside a thin plastic bag, sealed by masking tape and stored at 4° C. for 1-3 days. It is important that tassels are not stored beyond 3 days before further processing because longer storage may be detrimental. The selected tassels should contain microspores at an appropriate stage of development. In general, developing microspores that have at least completed meiosis are useful in the practice of the present invention. In a preferred embodiment, most microspores enclosed within the anthers are in the late uninucleate to early binucleate stages of development. Morphological features of tassels containing microspores at these stages can easily be established for each plant variety by comparing the morphology of the plant with the microspore developmental stage as determined by microscopic examination with acetocarmine stain or with 0.3 M mannitol solution. The stages of microspore development are set forth in Bennett, M.D. et al., Philosophical Transactions of the Royal Society (Lond.), B issue, 266:39-81 (1973), which is incorporated herein by reference. The morphology of a maize tassel is set forth in the following publication, which is incorporated herein by reference (How a Corn Plant Develops, Special Report No. 48, Iowa State University of Science and Technology Cooperative Extension Service, Ames, Iowa, June 1993).

The selected plant material, and therefore the microspores, is incubated in incubation medium at a temperature and osmolarity effective to induce androgenesis. The selected plant material can be completely or partially immersed in incubation medium and incubated at an effective temperature. In some embodiments, the selected plant material, and therefore the microspores, is incubated at a temperature between about 1° C. above the freezing point of the incubation medium to about 17° C. In some embodiments, the selected plant material, and therefore the microspores, is incubated at a temperature between about 4° C. and about 10° C. In some embodiments, the selected plant material, and therefore the microspores, is incubated at a temperature between about 8° C. and about 10° C. The duration of the temperature treatment is from about 3 days to about 21 days, such as from about 7 days to about 15 days, or from about 10 days to about 14 days. There is a relationship between the incubation temperature and the optimum duration of the temperature treatment; generally the lower the temperature the shorter the duration. For the M110 genotype, the preferred duration at 4° C. is from about 1 day to about 3 days, and the preferred duration at 9° C. is from about 7 days to about 17 days. The optimum temperature and duration of the temperature treatment varies with the genotype, but can be readily determined by one of ordinary skill in the art without undue experimentation.

The osmolarity of the incubation medium is typically from about 300 mOsm to about 450 mOsm. A representative incubation medium is MMA′, the composition of which is set out in Table 1. In some embodiments, the incubation medium contains an effective amount of at least one sporophytic development inducer, which induces microspores to switch from gametophytic to sporophytic development. By way of non-limiting example, sporophytic development inducers useful in the practice of the present invention may cause the development of inviable pollen grains, multicellular or multinucleate pollen grains, arrest starch formation in developing microspores, and cause physical deformation of mature pollen grains that develop from microspores treated with a sporophytic development inducer. Many sporophytic development inducers useful in the practice of the present invention are chemical hybridizing agents. Chemical hybridizing agents are chemicals which when applied to plants cause the plants to produce inviable pollen. Sporophytic development inducers useful in the present invention include, but are not limited to: amiprophos methyl, 2-aminonicotinic acid; 2-chloronicotinic acid; 6-chloronicotinic acid; 2-hydroxynicotinic acid; 6-hydroxynicotinic acid; 3-hydroxypicolinic acid; Benzotriazole; 2,2′-dipyridil; 2,4-pyridine dicarboxylic acid monohydrate; 2-hydroxypyridine; 2,3-dihydroxypyridine; 2,4-dihydroxypyrimidine-5-carboxylic acid; 2,4-dihydroxypyrimidine-5-carboxylic acid hydrate; dinitroaniline, phosphoric amide, 2-hydroxypirimidine hydrate; 2,4,5-trihydroxypyrimidine; 2,4,6-trichloropyrimidine; 2-hydroxy-4-methyl pyrimidine hydrochloride; 4-hydroxypyrazolo-3,4,d-pyrimidine; quinaldic acid; violuric acid monohydrate; thymine; xanthine; salicylic acid; sodium salicylate; salicyl aldehyde; salicyl hydrazide; 3-chlorosalicylic acid; fusaric acid; picolinic acid; butanediene monoxime; di-2-pyridyl ketone; salicin; 2,2′-dipyridil amine; 2,3,5-triiodobenzoic; 2-hydroxy pyridine-N-oxide; 2-hydroxy-3-nitropyridine; benzotriazole carboxylic acid; salicyl aldoxime; glycine; DL-histidine; penicillamine; 4-chlorosalicylic acid; 6-aminonicotinic acid; 2,3,5,6-tetrachloride 4-pyridine carboxylic acid; alpha benzoin oxime; 2,3-butadiene dioxime; isonicotinic hydrazide; cupferron; ethyl xanthic acid; 3-hydroxy benzyl alcohol; salicyl amide; salicyl anhydride; salicyl hydroxamic acid; methyl picolinic acid; 2-chloro pyridine; 2,6-pyridine carboxylic acid; 2,3-pyridine dicarboxylic acid; 2,5-pyridine dicarboxylic acid; Monsanto pyridones sold under the trade names Fenridazon and Genesis; pichloram; ammonium thiocyanate; amiben; diethyl dithiocarbamate; glyphosate; anthranilic acid; thiourea; 2,4-diclorophenoxyacetic acid; 4-chloro anisole; 2,3-dichloroanisole; 2-(2,4)-dichlorophenoxy propionic acid; 2-(4-chlorophenoxy)-2-methyl propionic acid; 2-(para-chloro phenoxy) isobutyric acid and α,β-dichlorobutyric acid. The effective concentration range of sporophytic development inducer is from about 0.001 mg/l to about 1000 mg/l, such as from about 1 mg/l to about 500 mg/l.

While not wishing to be bound to a particular theory explaining the method of action of the sporophytic development inducers useful in the practice of the present invention, representative sporophytic development inducers have some metal chelation ability. In particular, the foregoing, representative sporophytic development inducers can chelate Cu, Mg, Fe and Zn ions. Copper is essential to pollen fertility (Scharrer, K., and Schaumlaufel, E., Z. Plans. Dung. Bodenk, 89:1-17 (1960); see also, Tomasik, P. and Ratajewicz, Z., In: Newkome, G. R., and Strekowski, L., (eds.) Chapter 3, Pyridine-metal complexes, pp. 186-409 (1986)).

The sporophytic development inducers interact with the temperature treatment to enhance the induction of androgenic microspores. In addition, the sporophytic development inducers contribute to the completion of androgenesis leading to the eventual formation of mature embryoids, calli, or other regenerative tissue capable of regenerating into mature plants. Further, it will be understood that the sporophytic development inducers, temperature conditions, and incubation media described herein act synergistically to produce regenerative maize tissue from microspores.

Optionally, the temperature treatment of microspores occurs under conditions of nutrient stress. Nutrient stress may be effected by utilizing, for example in the incubation medium, an amount of at least one nutrient that is less than the amount of that nutrient necessary for the normal growth and development of the microspores in the incubation medium. Nutrient stress is one way in which to promote the induction of sporophytic development from microspores and can be used, for example, when dealing with microspores from maize genotypes that are resistant to androgenic induction.

In the practice of this aspect of the present invention, the temperature-treated microspores are isolated by any useful means. The temperature-treated microspores can be isolated, for example, by macerating the temperature-treated plant tissue, filtering the macerated plant tissue and separating viable temperature-treated microspores from other plant material. In some embodiments, the filtrate is subjected to density centrifugation, for example utilizing a solution of percoll, ficoll or mannitol, preferably a 0.3 M mannitol solution, layered over a higher density solution of percoll, ficoll, polyethylene glycol, or a sugar, preferably maltose, most preferably 0.58 M maltose.

The isolated, temperature-treated microspores are cultured under controlled osmolarity conditions in a cultivation medium containing at least one live plant ovary and/or ovary-conditioned medium, until the microspores develop into regenerative maize tissue, such as embryoids or calli. Ovary-conditioned medium is medium in which one or more live plant ovaries have been incubated, and which includes one or more chemical substances released by the ovary, or ovaries, that promote switching microspores from gametophytic to sporophytic development. In one embodiment, about 4 to about 6 live plant ovaries are added to a 60 mm diameter Petri dish containing approximately 5 ml of cultivation medium. Typically, live ovaries are replaced after about four weeks.

In some embodiments, the ovaries are obtained from wheat, such as cultivars Pavon 76 and Chris. In some embodiments, ovaries from plants other than wheat, such as barley or oats, are used.

The osmolarity of the cultivation medium is controlled so that it remains between about 300 mOsm and about 500 mOsm. The osmolarity of the cultivation medium can be controlled, for example, by periodically replacing, or supplementing, the existing cultivation medium with fresh cultivation medium. In some embodiments, the cultivation medium is maintained at an appropriate osmolarity by refreshing the cultivation medium at least once at about one week after culture initiation. The medium refreshment serves to remove toxic substances released by dead or degenerated microspores and/or to prevent excess change in medium osmolarity, normally resulting from the breakdown of sucrose by enzymes of dividing microspores. In some embodiments, the osmolarity is maintained within a suitable range by including a combination of sucrose and maltose as the source of carbon in the incubation medium.

Some embodiments of the cultivation medium include an effective amount of at least one auxin. Representative examples of auxins useful in the practice of the present invention include, but are not limited to: 2,4-dichlorophenoxyacetic acid (2,4-D), indoleacetic acid (IAA), indolebutyric acid (IBA), naphthalene acetic acid (NAA), and phenylacetic acid (PAA). The presently preferred concentration range for auxins in the cultivation medium is from about 0.01 mg/l to about 25 mg/l, such as from about 0.2 mg/l to about 10 mg/l, such as from about 0.5 mg/l to about 4.0 mg/l, such as from about 1.2 to about 2.5 mg/l.

In some embodiments, the cultivation medium includes an amount of at least one cytokinin effective to improve the quality of regenerative tissue, in particular to enhance the ability of regenerative tissue to grow and to increase the size to which regenerative tissue develops. Representative examples of cytokinins useful in the practice of the present invention include, but are not limited to: kinetin, benzaminopurine (BAP) and zeatin. Additionally, water in which peeled Solanum tuberosum potatoes have been boiled contains significant amounts of cytokinin(s) which can be utilized in the practice of the present invention. The presently preferred concentration range for kinetin, zeatin and BAP is from about 0.01 mg/l to about 10 mg/l, such as from about 0.2 mg/l to about 4.0 mg/l, or such as from about 0.5 mg/l to about 2.0 mg/l. A representative cultivation medium is IND, the composition of which is set forth in Table 3 herein.

Culture of temperature-treated microspores in cultivation medium yields regenerative maize tissue, such as embryoids, pro-embryoids and calli. Typically, the first cell divisions start at about 3 days after culture initiation. Multi-cellular structures typically are clearly defined after one week in culture. Pro-embryoids typically emerge out of the exine at about 7 to about 14 days after culture initiation. The first group of embryoids and/or calli typically becomes visible to the eye at about 21 days after culture initiation. The regenerative maize tissue may be regenerated into mature maize plants. In some embodiments, regenerative maize tissue that has reached the size of 2-3 mm in diameter is transferred to regeneration media to allow plant regeneration.

In some embodiments, regenerative maize tissue is transferred directly to a shoot regeneration medium. Typically, regenerative tissue on shoot regeneration medium is kept under light for two weeks. Some embodiments of the shoot regeneration medium include an amount of a cytokinin effective to improve the quality of regenerative tissue. Representative examples of useful cytokinins include, but are not limited to: kinetin, benzaminopurine (BAP) and zeatin. The presently preferred concentration range for kinetin, zeatin and BAP are as described above for the cultivation medium.

Another embodiment of the shoot regeneration medium includes an amount of an auxin effective to maintain callus development. Representative examples of useful auxins include, but are not limited to: 2,4-dichlorophenoxyacetic acid (2,4-D), indoleacetic acid (IAA), indolebutyric acid (IBA), and naphthalene acetic acid (NAA). The presently preferred concentration range for auxin in shoot regeneration medium is as described above for the cultivation medium. A representative shoot regeneration medium is Reg-II, the composition of which is provided in Table 3.

Once shoots grow to approximately 2 to 3 cm in height, regenerative maize tissue is typically transferred to a root regeneration medium. Some embodiments of the root regeneration medium are identical to the shoot regeneration media, but without auxins or cytokinins. A representative root regeneration medium is Reg-III, the composition of which is provided in Table 3. At about 7 days to about 10 days following the transfer to root regeneration medium, rooted regenerated plantlets are typically ready for transfer to a greenhouse or growth chamber for further growth.

Regenerative tissue of poor quality (for example, calli that appear loose and white in color) can be first transferred to a competency medium prior to transfer to regeneration medium. Typically, regenerative tissue is kept in the dark at 28° C. for about 1 to about 2 weeks following transfer to a competency medium and are then transferred to regeneration medium. Some embodiments of the competency medium include an amount of auxin effective to induce embryonic competency (i.e., the potential to become regenerable embryoids). Representative examples of useful auxins include, but are not limited to: 2,4-dichlorophenoxyacetic acid (2,4-D), indoleacetic acid (IAA), indolebutyric acid (IBA), and naphthalene acetic acid (NAA). In some embodiments, the competency medium is supplemented with 2,4-dichlorophenoxyacetic acid (2,4-D). In some embodiments, the competency medium is supplemented with PAA. In some embodiments, the competency medium is supplemented with 2,4-D and PAA. The presently preferred concentration range for auxins in competency medium is as described above for the cultivation medium. A representative competency medium is Reg-I, the composition of which is provided in Table 3.

Optionally, the microspores can be contacted with a spindle inhibiting agent, such as pronamide, or a gibberellin before, during, after, or overlapping with any portion of the temperature treatment.

Plants produced in accordance with the methods of the present invention can be doubled haploids. Additionally, plants produced in accordance with the methods of the present invention can be haploids, the chromosome number of which can subsequently be doubled by treatment with spindle inhibiting agents such as colchicines or caffeine or pronamide.

The methods of the present invention can be applied to any maize genotype including, but not limited to: A, M101, M102, M103, M104, M105, M106, M107, M108 and M110.

The methods of the present invention permit the production of plants from maize varieties and cultivars that have previously been considered recalcitrant or non-responsive to anther or microspore culture (for example, P-1, P-3 and P-4). An alternative solution to the problem of maize varieties, inbreds, or hybrids that are recalcitrant or non-responsive to anther or microspore culture, is to make crosses between the recalcitrant cultivars and cultivars that efficiently produce green plants from embryoids. In this approach, the methods of the present invention can be incorporated into a more general plant breeding program in which genotypes that are amenable to culture according to the methods of the present invention are crossed with less amenable genotypes which have other, desirable characteristics. The strategy of crossing a genotype that is amenable to the production of green, doubled haploid plants with a more recalcitrant cultivar, having some other desirable trait(s), is generally applicable to any maize variety, inbred, or hybrid.

Maize plants that are used to provide the microspore starting material in the practice of the methods of the present invention may be cultivated in the field, but preferably are cultivated in an artificial environment less exposed to microorganisms, such as a greenhouse. Field-grown maize plants are often heavily infested with microorganisms, which contaminate all stages of the microspore embryogenic process unless an effective disinfectant treatment is used. For example, the starting plant material used in the methods of the present invention can be treated with a 20% (v/v) solution of commercial hypochlorite or chlorine bleach. Any standard growth regime that is known to one of ordinary skill in the art for growing maize, preferably in a greenhouse, can be utilized in the practice of the present invention.

In some embodiments of the methods of the invention for producing maize plants from maize microspores, at least 300 embryoids and/or calli are obtained from seventy thousand microspores. In some embodiments, at least 190 green plants are regenerated from 150 embryoids or calli. In some embodiments, about 60% of healthy green plants regenerated are doubled haploids.

In another aspect of the present invention, methods are provided for producing regenerative maize tissue from maize microspores. The methods include the steps of: selecting plant material including microspores at a developmental stage amenable to androgenic induction; incubating the microspores in incubation medium at a temperature and osmolarity effective to induce androgenesis; isolating the temperature-treated microspores; and cultivating the isolated, temperature-treated microspores in cultivation medium containing at least one cytokinin and at least one auxin, and having an osmolarity between about 300 mOsm and about 500 mOsm, with at least one live plant ovary and/or ovary-conditioned medium to produce regenerative maize tissue.

The foregoing description of the methods of the invention for producing maize plants from maize microspores applies to the methods of this aspect of the invention, except that the methods of the invention for producing regenerative maize tissue from microspores do not include the step of regenerating maize plants from the regenerative maize tissue. In some embodiments of the methods of the invention for producing regenerative maize tissue from maize microspores, at least 300 embryoids and/or calli are obtained from seventy thousand microspores. Maize regenerative tissue produced according to the methods of the invention can be used, for example, to regenerate maize plants.

Microspores (preferably uninucleate microspores) treated in accordance with the methods of the present invention can optionally be genetically transformed by any art-recognized means in order to produce plants that express one or more desirable proteins. Examples of techniques for introducing a gene, cDNA, or other nucleic acid molecule into microspores include: transformation by means of Agrobacterium tumifaciens; electroporation-facilitated DNA uptake in which an electrical pulse transiently permeabilizes cell membranes, permitting the uptake of a variety of biological molecules, including recombinant DNA, by microspores; microinjection of nucleic acid molecules directly into microspores; treatment of microspores with polyethylene glycol; and bombardment of cells with DNA-laden microprojectiles which are propelled by explosive force or compressed gas to penetrate the microspore and enter the cell nucleus.

An example of a microspore transformation technique that utilizes Agrobacterium tumifaciens and is broadly applicable to numerous plant species is disclosed in European Patent Application EP 0 737 748 A1. Isolated microspores are cocultivated with Agrobacterium containing a Ti plasmid including a transgene (within the transfer DNA of the Ti plasmid) that is to be transferred and stably integrated into the microspore genome. Cellulytic enzymes (such as cellulase, hemicellulase and pectinase) are added during the cocultivation step and serve to permeabilize the microspore cell wall. The transfer DNA (T DNA) is transferred from the Agrobacterium cells to the microspores where it is inserted into the microspore genome thereby generating stably genetically transformed microspores. Thereafter, the treated microspores are washed with a mucolytic enzyme (such as lysozyme). Whole plants can then be regenerated from the genetically transformed microspores in accordance with the present invention. Other workers have reported the use of Agrobacterium to successfully transform microspores of Brassica (Pechan P. M., Plant Cell Rep. 8:387-390 (1989); Swanson E. B. and Erickson L. R., Theor. Appl. Genet. 78:831-835 (1989)).

An example of electroporation-facilitated permeabilization of microspores is reported in Joersbo et al., Plant Cell, Tissue and Organ Culture 23:125-129 (1990). Joersbo et al. report the transient electropermeabilization of barley microspores to the dye propidium iodide by delivering rectangular electrical pulses to microspores in a chamber with cylindrical coaxial electrodes at a distance of 1 mm. The electroporation treatment had limited deleterious effect on the microspores which could be cultured to produce green plants. Similarly, Fennell and Hauptmann (Plant Cell Reports 11:567-570 (1992)) reported the electroporation-mediated delivery of plasmid DNA into maize microspores, and also reported the polyethylene glycol (PEG)-mediated delivery of plasmid DNA into maize microspores.

Another method for stably genetically transforming microspores is biolistic transformation whereby microspores are bombarded with DNA-laden microprojectiles which are propelled by explosive force or compressed gas to penetrate the microspore. Yao et al. (Genome 40(4):570-581 (1997)) report the production of transgenic barley plants by direct delivery of plasmid DNA into isolated microspores using high velocity microprojectiles. The plasmid used to transform the microspores contained a bar gene, under the control of a maize ubiquitin promoter, that conferred resistance to the herbicide bialaphos. Thus, genetically transformed microspores or embryoids could be selected based on their resistance to bialaphos present in the culture medium. Similarly, Jahne et al. (Theor. Appl Genet. 89:525-533 (1994)) also report the production of transgenic barley plants by direct delivery of plasmid DNA into isolated microspores using high velocity gold microprojectiles. Again, genetically transformed microspores or microspore-derived calli were selected based on their resistance to bialaphos present in the culture medium. Fukuoka et al. (Plant Cell Reports 17:323-328 (1998)) report the production of transgenic rapeseed plants by direct delivery of plasmid DNA into isolated microspores using high velocity microprojectiles. Transformed embryos derived from the microprojectile bombarded microspores were identified by expression of a firefly luciferase gene. Harwood et al. (Euphytica 85:113-118 (1995)) disclose the use of the PDS1000 He particle delivery system to genetically transform barley microspores. The gus reporter gene was used to demonstrate both transient and stable transformation events. Additional examples of microspore transformation techniques are set forth in In Vitro Haploid Production in Higher Plants, Chapt. 2, Jain et al. (eds.), Kluwer Academic Publishers (1996). The aforementioned publications disclosing microspore transformation techniques are incorporated herein by reference, and minor variations make these technologies applicable to a broad range of maize plants.

The following examples merely illustrate the best mode now contemplated for practicing the invention, but should not be construed to limit the invention.

### EXAMPLE 1

### Generating Maize Plants From Microspores

Growing Maize Plants.

One seed is sown into pre-mixed soil in each 10×12 inch pot. Plants are grown in either a greenhouse or growth chamber with a day/night temperature regime of 30° C./20° C., and a photoperiod of 16/8 hr. Fertilizers with N:P:K ratio of 20:20:20 are pre-mixed with soil. Additional fertilizer with the same nutrient ratio is provided through daily watering. No exceptional growing condition is necessary so long as healthy plants are raised.

Collecting Tassels.

For every genotype or hybrid, a correlation between plant morphology and developmental stage of microspores is established through a microscopic examination. Before the tassel enclosed in the boot is about to emerge, 2 to 3 florets on the top of a tassel are picked out by a pair of long forceps, with one hand holding the base of the boot and plant firmly. Sampled florets are brought back to the laboratory where anthers are taken out of florets and crushed with a glass rod in a drop of acetocarmine or 0.3 M mannitol solution on a slide. The developmental stage of the microspores on the slide is then scored under an inverted or a light microscope. If most microspores are at late-uninucleate to early binucleate stages, the whole tassel is ready to be sampled. Once established for each genotype/hybrid, the relative location between the boot and the enclosed tassel can be used as a convenient criterion for sampling. Plants are cut at 1 to 2 nodes below the tassel base. All foliage is removed, except 3 to 4 leaves outside the tassel. These 3 to 4 leaves are trimmed to just 1 inch longer than the tassel itself and the tassel is placed in a flask (or other container) with the base in contact with distilled water in the flask. The flask is then brought back to the laboratory for further processing. Tassels so harvested can be disinfected immediately when the schedule permits, or stored in a refrigerator at 4° C. For storage, the flask or container with a tassel is wrapped in a plastic grocery vegetable bag, which is then sealed by masking tape. Tassels may be stored this way for 1 to 3 days with no, or minimal, detrimental effects on microspore viability. However, storage beyond 3 days without further processing may be detrimental, and hence is strongly discouraged.

Temperature Treatment of Tassels.

In a laminar flow hood, the remaining foliage encasing the tassel is removed and the tassel is then taken out of the boot for disinfection. The tassels are disinfected as a whole or first separated into florets for disinfection. The whole tassel is submerged into a 20% commercial bleach (sodium hypochlorite) solution in a graduated cylinder for 20 min, during which periodic shaking is applied. The bleach solution is then poured out, followed by three rinses with autoclaved distilled water. The florets are then removed with two pairs of forceps and placed into a 100 mm diameter Petri dish for temperature treatment. If florets are separated first and then disinfected, they are transferred directly to a 100 mm Petri dish for temperature treatment after the disinfection step, which is identical to the handling of a whole tassel.

In each 100 mm Petri dish, 150-200 florets are floated over 10-15 ml of MMA′ medium (Table 1) including an inert sugar (mannitol) and at least one sporophytic development inducer, which switches microspores from gametophytic to sporophytic development. Petri dishes are sealed with parafilm and placed in incubators with temperature ranging from 4° to 10° (+/−1° C.) in the dark for 8 to 14 days.

TABLE 1
MMA′ for Temperature Treatment of Tassels or Florets

| Component | mg/L |
|---|---|
| KCl | 1,492 |
| MgSO4-7H₂O | 246 |
| CaCl₂-2H₂O | 148 |
| KH₂PO₄ | 136 |
| H₃BO₃ | 3 |
| KI | 0.5 |
| MnSO₄-H₂O | 8 |
| ZnSO₄-7H₂O | 0.3 |
| Fe-NaEDTA | 56 |
| Mannitol | 54,700 |
| Ascorbic acid | 50 |
| 2-HNA | 100 |

Microspore Isolation.

At the completion of the temperature treatment, Petri dishes are brought to a laminar flow hood, and florets in the Petri dishes are transferred into an MC-II Waring blender cup. Fifty to sixty milliliters of isolation medium (Table 2) or 0.3 M mannitol or 6% maltose plus 50 mg/l ascorbic acid is added to the blender cup. The florets are blended at 14,000 rpm for 10 seconds, and at 16,000 rpm for seconds. The blender cups are transferred back to the laminar flow hood and the slurry is filtered through a 100 μm mesh filter. The filtrate is collected and filtered again through a 50 μm mesh filter. Microspores retained on top of the 50 μm mesh filter are rinsed three times with 2 ml of 0.3 M mannitol solution and then washed off the filter and into a 60 mm diameter Petri dish with 2 ml of 0.3 M mannitol solution.

The mixture of microspores and 0.3 M mannitol solution is then layered over 5 ml of 18-21% maltose in a sterile 15 ml conical centrifuge tube. The tube is balanced off and centrifuged for 2 minutes at 750 rpm. Viable microspores form one or two band(s) at the top of the 18-21% maltose, while debris and damaged microspores form a pellet at the bottom of the tube.

TABLE 2
Isolation Medium

| Component | Concentration (mg/L) |
|---|---|
| Ascorbic Acid | 50 |
| Biotin | 0.1 |
| Nicotinic Acid | 10 |
| Proline | 100 |
| Mannitol | 54,700 |
| Adjust pH to 5.7-6.0 | |

The band(s) plus 3 ml supernatant are transferred into another 15 ml conical centrifuge tube and centrifuged at 1,400 rpm for 1.5 minutes. The microspores form a band on top of the 18-21% maltose solution. The microspores in the band are carefully collected and transferred with a pipette to another 50 μm mesh filter. The solution is allowed to pass through while microspores are retained on top of the mesh filter. The microspores trapped in the mesh filter are rinsed three times with 2 ml each of cultivation medium IND (Table 3). Microspores are then rinsed off the mesh filter and into a 20×60 mm Petri dish with 2 ml of cultivation medium IND. Microspore density is assessed through a haemocytometer under an inverted microscope, and the microspores are evenly divided into Petri dishes for cultivation. In all cultures, the density of microspores is made approximately 7×10⁴/ml.

TABLE 3
Medium Recipes for Cultivation and Plant Regeneration

| Chemical (mg/L) | IND | Reg-I | Reg-II | Reg-III |
|---|---|---|---|---|
| (NH₄)₂SO₄ | | 463 | | |
| KNO₃ | 2500 | 2830 | 2500 | 2500 |
| CaCl₂-2H₂O | 176 | 166 | 176 | 176 |
| NH₄NO₃ | 165 | | 165 | 165 |
| KH₂PO₄ | 510 | 400 | 510 | 510 |
| MgSO₄-7H₂O | 370 | 185 | 370 | 370 |
| Na-EDTA | 37.3 | | 37.3 | 37.3 |
| FeSO₄-7H₂O | 27.8 | 27.8 | 27.8 | |
| H₃BO₃ | 1.6 | 1.6 | 1.6 | 1.6 |
| KI | 0.8 | 0.8 | 0.8 | 0.8 |
| MnSO₄-4 H₂O | 4.4 | 4.4 | 4.4 | 4.4 |
| ZnSO₄-7 H₂O | 1.5 | 1.5 | 1.5 | 1.5 |
| Myo-inositol | | 100 | | |
| Glycine | 2.0 | | 2.0 | 2.0 |
| Nicotinic Acid | 0.5 | 1.0 | 0.5 | 0.5 |
| Pyridoxine | | 1.0 | | |
| L-Proline | 400 | 2880 | | |
| Thiamine-HCl | 0.5 | 10 | 1.0 | 1.0 |
| Asparagine | 15 | | | |
| Sucrose | 50,000 | 30,000 | 30,000 | 30,000 |
| Maltose | 70,000 | | | |
| Casein | | 100 | | |
| hydrolysate | | | | |
| Glutamine | 125 | | 146 | 146 |
| BAP | | | 2.0 | |
| Kinetin | 0.4 | | 2.0 | |
| 2,4-D | 1.2 | 2.0 | | |
| PAA | 1.0 | | | |
| NAA | | | 1.0 | |
| Gelrite | | 2,700 | 4,000 | 4,000 |

Cultivation of Microspores.

4 to 6 wheat ovaries are added into each Petri dish with isolated maize microspores. All Petri dishes are sealed with parafilm and placed in an incubator in the dark with a preset temperature of 27° to 28° C. The cultivation medium is refreshed one week after the culture initiation, and wheat ovaries are replaced at four weeks. The medium refreshment serves to remove toxic substances that were released by dead or degenerated microspores and/or prevent excess change in medium osmolarity, normally resulting from the breakdown of sucrose by enzymes of dividing microspores. During this culture period, the culture is closely monitored. The first cell divisions typically start after 3 days in culture. Multi-cellular structures typically are clearly defined after one week in culture. Pro-embryoids emerge out of the exine in about 11 to 14 days following the culture initiation. The first group of embryoids/calli becomes visible to the eye approximately 21 days from the culture initiation. Once embryoids/calli reach the size of 2 to 3 mm in diameter, they are transferred to regeneration media to allow direct or indirect plant regeneration.

Regeneration of Maize Plants.

Regenerative tissues of good quality, which are yellowish and compact, are transferred directly to shoot regeneration medium Reg-II (Table 3) for plant regeneration. After shoots have emerged, regenerating embryoids or calli are transferred to root regeneration medium Reg-III. Regenerative tissues of poor quality (i.e., that appear loose and white in color) can be first transferred to competency medium Reg-I (Table 3) to induce embryogenic competency. Following such transfer, regenerative tissues are kept in the dark at 28° C. for 1 to 2 weeks before transfer to shoot regeneration medium Reg-II for plant development. Petri dishes with regenerative tissue on Reg-II are kept under light for two weeks. Once shoots grow to approximately 2 to 3 cm in height, they are transferred to tissue culture tubes containing root regeneration medium Reg-II (Table 3), for root initiation. About 7 to 10 days following the transfer to root regeneration medium Reg-III, regenerated plantlets are ready for transfer to a greenhouse or growth chamber for further growth to facilitate the examination of chromosome doubling or seed production.

### EXAMPLE 2

### Optimization of Cultivation Conditions

Basic Medium Component.

Macro- or micro-nutrients in NPB 99, Yu-Pei, Zheng's 14, and N6 are all suitable for use in the cultivation medium (Table 4). A preferred medium is a modified Yu-Pei medium (IND, Table 3).

Carbon Source.

Among the maize genotypes that were tested, M104 and M105 responded to 9 to 12% maltose. Genotypes A, M101, M103 and M 110 responded better to sucrose. For genotype M110, the best carbon source was a combination of sucrose with maltose, for a total concentration of 9% or 12%. With a combination of 5% sucrose and 7% maltose, or 2% sucrose and 4% maltose in the cultivation medium, it is possible to obtain about 300 mature embryoids and/or calli from a Petri dish containing a total of 7×10⁴ microspores. This combination keeps the sucrose concentration to the functional minimal and uses maltose as the more stable osmoticum. Media with such sugar combinations exhibit acceptable fluctuations in osmolarity (Tables 5-7).

TABLE 4
Medium Composition of NPB 99, Yu-Pei, Zheng’s 14, and N6

| Chemical (mg/L) | NPB-99 | Zheng 14 | Yu-Pei | N-6 |
|---|---|---|---|---|
| (NH₄)₂SO₄ | 232 | 150 | | 464 |
| KNO₃ | 1415 | 3000 | 2500 | 2830 |
| CaCl₂-2H₂O | 83 | 150 | 176 | 166 |
| NH₄NO₃ | | | 165 | |
| KH₂PO₄ | 200 | 600 | 510 | 400 |
| MgSO₄-7H₂O | 93 | 450 | 370 | 186 |
| EDTA | 37.3 | 37.3 | 37.3 | 37.3 |
| FeSO₄-7H₂O | 27.8 | 27.8 | 27.8 | 27.8 |
| H₃BO₃ | 5 | 3 | 1.6 | 10 |
| CoCl-6 H₂O | 0.0125 | 0.025 | | 0.0125 |
| CuSO₄-5 H₂O | 0.0125 | 0.025 | | 0.0125 |
| KI | 0.4 | 0.75 | 0.8 | 0.4 |
| MnSO₄-4 H₂O | 5 | 10 | 4.4 | 5 |
| NaMoO₄-2 H₂O | 0.0125 | 0.25 | | 0.0125 |
| ZnSO₄-7 H₂O | 5 | 2 | 1.5 | 5 |
| Myo inositol | 50 | 100 | | 50 |
| Glycine | | 2 | 7.7 | |
| Nicotinic Acid | 0.5 | 1 | 1 | 0.5 |
| Pyridoxine-HCl | 0.5 | 1 | | 0.5 |
| Thiamine-HCl | 5 | 10 | 0.25 | 5 |
| Sucrose | | 150,000 | | |
| Maltose | 90,000 | | 120,000 | 90,000 |
| Casein hydrolysate | | 500 | 500 | |
| Glutamine | 500 | | | 500 |
| 6-BA | | 1 | 1 | |
| Kinetin | 0.2 | | | |
| 2,4-D | 0.2 | 2 | 0.5 | |
| PAA | 1 | | 1.0 | |
| NAA | | 1 | | |

It was observed that media with maltose as the sole carbon source experience very little change in osmolarity over regenerative tissue development time, while the opposite is true for media with sucrose as the only carbon source. Such osmolarity changes are particularly common with autoclave-sterilized sucrose-containing media. Convincing evidence has been accumulated for a positive role of sucrose in promoting cell divisions in maize microspore cultures. To reconcile the use of sucrose and the change of osmolarity in the medium, it is essential to study the correlation between sugar concentrations and changes in osmolarity over a period of time. The following experiments were designed to test the changes in osmolarity and pH for media with various combinations of sucrose and maltose. Genotype M110 was grown in the NPB greenhouse. Tassels were sampled when the top florets were at the late uninucleate stage. The procedures for disinfection, temperature treatment, isolation and regenerative tissue cultivation were as described above in EXAMPLE 1

TABLE 5
Changes in Osmolarity and pH in Eight Media after 10 days of
Incubation

| | Sucrose: Maltose Ratio in Medium (12% Total Sugar) | | | | | | | |
|---|---|---|---|---|---|---|---|---|
| | 1:11 | 2:10 | 3:9 | 4:8 | 5:7 | 7:5 | 9:3 | 10:2 |
| Day 1 | 405 | 426 | 419 | 429 | 424 | 437 | 445 | 352 |
| Day 10 | 461 | 504 | 496 | 513 | 500 | 519 | 538 | 424 |
| Change | +56 | +78 | +77 | +84 | +76 | +82 | +93 | +72 |
| pH: | | | | | | | | |
| Day 1 | 5.69 | 5.78 | 5.79 | 5.80 | 5.73 | 5.80 | 5.80 | 5.95 |
| Day 10 | 4.85 | 4.75 | 4.94 | 4.91 | 5.13 | 5.14 | 4.91 | 5.72 |
| Change | −0.84 | −1.03 | −0.85 | −0.89 | −0.60 | −0.66 | −0.89 | −0.23 |

Except for sugar combinations indicated in Tables 5-7, all other medium components were the same as cultivation medium IND (Table 3). Even though all of the media were filter-sterilized, dramatic changes in osmolarity still occurred. With as low as 1% sucrose in the mixture, the change in osmolarity was still substantial over a time period of 21 days (Table 7). As sucrose concentration increases, so does the rise of osmolarity for media (Tables 5-7). Such increases in osmolarity, however, are non-linear and not directly proportional to increases in sucrose concentration ratios.

TABLE 6
Changes in Osmolarity and pH in Six Media after 14 days of
Incubation

| | Sucrose: Maltose Ratio in Medium (12% Total Sugar) | | | | | |
|---|---|---|---|---|---|---|
| | 0.5-11.5 | 1-11 | 2-10 | 3-9 | 4-8 | 5-7 |
| Osmolarity: | | | | | | |
| Day 1 | 392 | 403 | 426 | 419 | 429 | 428 |
| Day 14 | 434 | 587 | 527 | 524 | 559 | 583 |
| Change | 42 | 94 | 101 | 105 | 130 | 155 |
| pH: | | | | | | |
| Day 1 | 5.85 | 5.87 | 5.82 | 5.85 | 5.90 | 5.87 |
| Day 14 | 5.18 | 5.29 | 4.99 | 5.00 | 5.03 | 5.27 |
| Change | −0.67 | −0.58 | −0.83 | −0.85 | −0.87 | −0.60 |

From these tests, it was observed that the higher the concentration of sucrose, the larger the increase of osmolarity. The longer the media are left without refreshing, the higher the increases in osmolarity. In addition, the pH also drops to a more acidic level that is undesirable for microspore embryogenesis. Therefore, to prevent dramatic increase in osmolarity and decrease in pH, a combination of sucrose and maltose was used instead of pure sucrose, and the cultivation medium was refreshed periodically. The initial pH of the cultivation medium was also adjusted to around 6.0.

TABLE 7
Changes in Osmolarity and pH in Five Media after 21 days of
Cultivation

| | Sucrose: Maltose Ratio in Medium (12% Total Sugar) | | | | |
|---|---|---|---|---|---|
| | 1-11 | 2-10 | 3-9 | 4-8 | 5-7 |
| Osmolarity: | | | | | |
| Day 1 | 416 | 427 | 427 | 432 | 427 |
| Day 21 | 576 | 614 | 584 | 610 | 650 |
| Change | +160 | +187 | +155 | +183 | +223 |
| pH: | | | | | |
| Day 1 | 5.87 | 5.82 | 5.85 | 5.90 | 5.87 |
| Day 21 | 4.73 | 4.61 | 4.48 | 4.82 | 4.57 |
| Change | −1.14 | −1.21 | −1.37 | −1.08 | −1.30 |

Plant Growth Regulators:

It was found that various combinations of plant hormones work effectively for embryoid/callus cultivation. Among these, a combination of 2,4-D (1.2 mg/l), PAA (1.0 mg/l) and kinetin (0.4 mg/l) is most effective for regenerative tissue cultivation of genotype M110.

### EXAMPLE 3

### Optimization of Temperature Treatment Conditions

The temperature treatment conditions were optimized using various temperature regimes, a sporophyte development inducer, and various durations of temperature treatments. The results are presented in Tables 8-10. The temperature experiments set forth in Table 8 indicated that higher temperatures (27° C. and 32° C.) were not effective for inducing embryogenesis. In contrast, temperatures treatments at 4° C., 6° C., 9° C., and treatments at combinations of these temperatures, were found to be effective for triggering androgenesis.

TABLE 8
The Effect of Temperature Treatment Combined with 100 mg/l 2-HNA

| | Temperature treatments | | | | | | |
|---|---|---|---|---|---|---|---|
| | | | 4° C./ | | 4° C./ | | |
| Gen- | | | 3d & | | 3d & | | |
| o- | 4° C./ | 6° C./ | 6° C./ | 9° C./ | 9° C./ | 27° C./ | 32° C./ |
| type: | 7d | 10d | 7d | 14d | 8d | 1d | 1d |
| M101 | + | + | + | + | + | − | − |
| M102 | 0 | 0 | + | + | + | − | − |
| M103 | 0 | 0 | + | + | + | − | − |
| M104 | 0 | 0 | − | + | + | − | − |
| M105 | 0 | 0 | + | + | + | − | − |
| M110 | + | + | + | + | + | − | − |
| A | + | + | + | + | + | − | − |

Table Legend: “−” means there was no inducing effect observed; “+” means that there were inducing effects observed; “0” indicates that no test was done.

Table Legend: “−” means there was no inducing effect observed; “+” means that there were inducing effects observed; “0” indicates that no test was done.

The results of the experiments set forth in Table 8 indicate that there is a relationship between the treatment temperature and the optimum duration of the temperature treatment: the lower the temperature the shorter the treatment period (e.g., if the treatment is more than 9 days at 4° C., the percentage of viable microspores was less than 20%). Temperature treatment at 9° C. for 11 to 14 days gave better inducing effects. With this regime, over 35% microspores of genotype M110 were induced. More interestingly, temperature treatments at 4° C., 6° C., and at a combination of 4° C. and 6° C., or at a combination of 4° C. and 9° C., also facilitated induction of androgenesis. These temperatures have practical importance because the different treatment periods allow the isolation of microspores at different time points from the time of tassel harvest, easing the workload when too many tassels are available on a given day. These temperatures also may allow flexibility for a range of genotypes.

TABLE 9
Treatment Temperature and Length for Genotype M110

| Days of | | | | | | | | | |
|---|---|---|---|---|---|---|---|---|---|
| temperature | | | | | | | | | |
| treatment | 1 | 3 | 5 | 7 | 9 | 11 | 13 | 15 | 17 |
| 4° C. | | + | + | + | | | | | |
| 6° C. | | | + | + | + | + | | | |
| 9° C. | | | | + | + | + | + | + | + |

Table Legend: “+” means that inducing effects were observed.

Table 10 shows the percentages of viable cells and the percentages of induced cells obtained after treatment at 9° C. for various durations. Combining both factors (i.e., the percentage of viable cells and the percentage of induced cells obtained), the best treatment period for M110 at 9° C. is from about 9 to about 13 days.

TABLE 10
Percentages of Viable and Induced Cells Following Various
Temperature Treatment Periods at 9° C. for genotype M110

| Days of | | | | | | | | | |
|---|---|---|---|---|---|---|---|---|---|
| Temperature | | | | | | | | | |
| Treatment | 1 | 3 | 5 | 7 | 9 | 11 | 13 | 15 | 17 |
| Viable Cells (%) | 95 | 95 | 90 | 90 | 85 | 85 | 80 | 60 | 40 |
| Induced Cells (%) | | | | 20 | 30 | 35 | 37 | 36 | 25 |

For genotype M110, it is possible to induce embryogenic microspores using the temperature treatment described above. The induced microspores were characterized by the location of the cytoplasm in the cell center around the nucleus, and in some cases by the presence of thin cytoplasmic strands running towards the periphery of the cell. Dividing cells appeared after 3 to 4 days in culture, then continued to divide until 12 days in culture, during which the percentage of the multicellular structures increased to a maximum of 20%. Only about 4% of the multicellular structures ruptured their exine and emerged out of the cell wall. About 300 mature embryoids and/or calli were obtained from a population of 7×10⁴ (2 ml) microspores in culture. Although some calli/embryoids of poor quality did not regenerate into plants, others produced multiple plantlets. Up to 200 green plants were regenerated out of 150 regenerable embryoids/calli. Among healthy green plants regenerated from these embryoids/calli, about 60% were doubled haploids.

### EXAMPLE 4

### Experiments With Different Maize Genotypes

Microspores from several different maize genotypes were induced to produce regenerative maize tissue. M110 is a sweet corn cultivar released to the public. The percentages of dividing microspores, pro-embryoids, and mature embryoids obtained from M110 are summarized in Table 11.

TABLE 11
Percentage of Induced Microspores, Dividing Microspores,
Multicellular Structures, Pre-embryoids and Mature Embryoids
from Microspores of Genotype M110 in Cultivation Medium

| | Days in Culture | | | | | | | | |
|---|---|---|---|---|---|---|---|---|---|
| | 1- | 3- | 5- | 8- | 10- | 12- | 14- | 16- | 35- |
| % | 2 | 5 | 8 | 10 | 12 | 14 | 16 | 18 | 45 |
| Induced | 36 | | | | | | | | |
| Microspores | | | | | | | | | |
| Dividing | | 25 | | | | | | | |
| Microspore | | | | | | | | | |
| Multicellular | | | 18 | 18 | 20 | | | | |
| structures | | | | | | | | | |
| Pro- | | | | | | 3 | 4 | 4 | |
| embryoids | | | | | | | | | |
| Calli/mature | | | | | | | | | 300* |
| Embryoids | | | | | | | | | |

*This is the total number of calli/embryoids obtained from 7 × 10⁴ microspores.

In a preliminary experiment, immature tassels of four elite proprietary genotypes (P1, P2, P3 and P4) were shipped from remote growing location. This method of harvesting microspores probably reduced the overall response. Table 12 summarizes the responsiveness of P-1, P-3 and P-4 in terms of the percentages of induced microspores and dividing microspores obtained, and the number of embryoids obtained from 10⁴ microspores.

TABLE 12
Culture Responses by Elite Proprietary Genotypes

| | Induced | Dividing | Embryoids/Calli |
|---|---|---|---|
| | Microspore | Microspores | (number per 10⁴ |
| Genotype | (%) | (%) | microspores) |
| P-1 | 40 | 10 | 3 |
| P-3 | 37 | 8 | 17 |
| P-4 | 30 | 6 | 8 |

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. A method of producing maize plants from maize microspores, comprising:
(a) selecting maize plant material comprising maize microspores at a developmental stage amenable to androgenic induction;
(b) incubating the microspores in incubation medium at a temperature and osmolarity effective to induce androgenesis to obtain temperature-treated microspores;
(c) isolating the temperature-treated microspores;
(d) cultivating the isolated, temperature-treated microspores in cultivation medium with either at least one live plant ovary or ovary-conditioned medium to produce regenerative maize tissue, wherein the cultivation medium has an osmolarity between about 300 mOsm and about 500 mOsm and comprises at least one cytokinin and at least one auxin; and
(e) regenerating maize plants from the regenerative maize tissue.

2. The method of claim 1, wherein the plant material comprises at least one of tassels, florets, and anthers.

3. The method of claim 1, wherein the microspores are in the late-uninucleate to early-binucleate developmental stage.

4. The method of claim 1, wherein the microspores are incubated in incubation medium at a temperature from about 1° C. above the freezing temperature of the incubation medium to about 17° C.

5. The method of claim 1, wherein the microspores are incubated in incubation medium at a temperature from about 4° C. to about 10° C.

6. The method of claim 1, wherein the microspores are incubated in incubation medium at a temperature from about 8° C. to about 10° C.

7. The method of claim 1, wherein the microspores are incubated in incubation medium for about 3 days to about 21 days.

8. The method of claim 1, wherein the microspores are incubated in incubation medium for about 7 days to about 15 days.

9. The method of claim 1, wherein the microspores are incubated in incubation medium for about 10 days to about 14 days.

10. The method of claim 1, wherein the incubation medium has an osmolarity between about 300 mOsm and about 450 mOsm.

11. The method of claim 1, wherein the incubation medium comprises at least one sporophytic development inducer.

12. The method of claim 11, wherein the at least one sporophytic development inducer is selected from the group consisting of 2-aminonicotinic acid; 2-chloronicotinic acid; 6-chloronicotinic acid; 2-chloroethyl-phosphonic acid; 2-hydroxynicotinic acid; 6-hydroxynicotinic acid; 3-hydroxypicolinic acid; Benzotriazole; 2-hydroxyproline; 2,2′-dipyridil; 2,4-pyridine dicarboxylic acid monohydrate; 2-hydroxypyridine; 2,3-dihydroxypyridine; 2,4-dihydroxypyrimidine-5-carboxylic acid; 2,4-dihydroxypyrimidine-5-carboxylic acid hydrate; 2-hydroxypirimidine hydrate; 2,4,5-trihydroxypyrimidine; 2,4,6-trichloropyrimidine; 2-hydroxy-4-methyl pyrimidine hydrochloride; 4-hydroxypyrazolo-3,4,d-pyrimidine; quinaldic acid; violuric acid monohydrate; thymine; xanthine; salicylic acid; sodium salicylate; salicyl aldehyde; salicyl hydrazide; 3-chlorosalicylic acid; fusaric acid; picolinic acid; butanediene monoxime; di-2-pyridyl ketone; salicin; 2,2′-dipyridil amine; 2,3,5-triiodobenzoic; 2-hydroxy-pyridine-N-oxide; 2-hydroxy-3-nitropyridine; benzotriazole carboxylic acid; salicyl aldoxime; glycine; D L-histidine; penicillamine; 4-chlorosalicylic acid; 6-aminonicotinic acid; 2,3,5,6-tetrachloride 4-pyridine carboxylic acid; alpha benzoin oxime; 2,3-butadiene dioxime; isonicotinic hydrazide; cupferron; ethyl xanthic acid; 3-hydroxy benzyl alcohol; salicyl amide; salicyl anhydride; salicyl hydroxamic acid; methyl picolinic acid; 2-chloro pyridine; 2,6-pyridine carboxylic acid; 2,3-pyridine dicarboxylic acid; 2,5-pyridine dicarboxylic acid; pichloram; ammonium thiocyanate; amiben; diethyl dithiocarbamate; glyphosate; anthranilic acid; thiourea; 2,4-diclorophenoxyacetic acid; 4-chloro anisole; 2,3-dichloroanisole; 2-(2,4)-dichlorophenoxy propionic acid; 2-(4-chlorophenoxy)-2-methyl propionic acid; 2-(para-chloro phenoxy) isobutyric acid and α,β-dichlorobutyric acid.

13. The method of claim 11, wherein the at least one sporophytic development inducer is 2-hydroxynicotinic acid.

14. The method of claim 11, wherein the concentration of the at least one sporophytic development inducer is from about 0.001 mg/l to about 1000 mg/l.

15. The method of claim 11, wherein the concentration of the at least one sporophytic development inducer is from about 1 mg/l to about 500 mg/l.

16. The method of claim 1, wherein the incubation medium is MMA′.

17. The method of claim 1, wherein the incubation medium is an aqueous medium comprising an amount of at least one nutrient that is less than the amount of that nutrient necessary for the optimal growth and development of the microspores in the aqueous medium.

18. The method of claim 1, wherein the temperature-treated microspores are isolated using density centrifugation.

19. The method of claim 18 wherein the density centrifugation utilizes a solution of mannitol layered over a solution of maltose, wherein the solution of maltose has a higher density than the solution of mannitol.

20. The method of claim 1, wherein the cultivation medium comprises sucrose and maltose.

21. The method of claim 1, wherein the cultivation medium is periodically refreshed to maintain an osmolarity of the cultivation medium between about 300 mOsm and about 500 mOsm.

22. The method of claim 1, wherein the at least one auxin is selected from the group consisting of 2,4-dichlorophenoxyacetic acid, indoleacetic acid, indolebutyric acid, naphthalene acetic acid, and phenylacetic acid.

23. The method of claim 22, wherein the at least one auxin is present at a concentration from about 0.01 mg/l to about 25 mg/l.

24. The method of claim 22, wherein the at least one auxin is present at a concentration from about 0.2 mg/l to about 10 mg/l.

25. The method of claim 22, wherein the at least one auxin is present at a concentration from about 1.2 mg/l to about 2.5 mg/l.

26. The method of claim 1, wherein the at least one cytokinin is selected from the group consisting of kinetin, benzaminopurine, and zeatin.

27. The method of claim 26, wherein the concentration of the at least one cytokinin is from about 0.01 mg/l to about 10 mg/l.

28. The method of claim 26, wherein the concentration of the at least one cytokinin is from about 0.2 mg/l to about 4 mg/l.

29. The method of claim 26, wherein the concentration of the at least one cytokinin is from about 0.5 mg/l to about 2 mg/l.

30. The method of claim 1, wherein the cultivation medium is IND.

31. The method of claim 1, wherein the at least one live ovary is a wheat ovary.

32. The method of claim 1, wherein the at least one live ovary is a barley ovary.

33. The method of claim 32, wherein the at least one live ovary is a barley cultivar Igri ovary.

34. The method of claim 1 wherein the cultivating step utilizes ovary-conditioned medium.

35. The method of claim 1, wherein the step of regenerating maize plants comprises the steps of:
(a) culturing the regenerative maize tissue in shoot regeneration medium to produce shoot-containing regenerative maize tissue, wherein the shoot regeneration medium comprises at least one cytokinin and at least one auxin; and
(b) culturing the shoot-containing regenerative maize tissue in root regeneration medium to produce roots.

36. The method of claim 35, wherein the at least one cytokinin is kinetin.

37. The method of claim 35, wherein the concentration of the at least one cytokinin is 0.01 to 10 mg/l.

38. The method of claim 35, wherein the at least one auxin is napthalene acetic acid.

39. The method of claim 35, wherein the concentration of the at least one auxin is from about 0.01 mg/l to about 25 mg/l.

40. The method of claim 35, wherein the shoot regeneration medium is Reg-II.

41. The method of claim 35, wherein the root regeneration medium is Reg-III.

42. The method of claim 1, further comprising the step of transferring regenerative maize tissue to a competency medium prior to the maize plant regeneration step.

43. The method of claim 42, wherein the competency medium comprises at least one auxin.

44. The method of claim 42, wherein the competency medium is Reg-I.

45. The method of claim 1 further comprising the step of genetically transforming said microspores, the step of transformation being effected prior to the maize plant regeneration step.

46. Genetically transformed plants produced according to the method of claim 45.

47. A method of producing maize plants from maize microspores comprising:
(a) selecting maize tassels or florets comprising microspores at a developmental stage amenable to androgenic induction;
(b) incubating the microspores in incubation medium with an effective amount of a sporophytic development inducer at around 8° C. to around 10° C. for about days to about 14 days to obtain temperature-treated microspores, wherein the incubation medium has an osmolarity between about 300 mOsm and about 450 mOsm and comprises 2-hydroxynicotinic acid;
(c) isolating the temperature-treated microspores;
(d) cultivating the isolated, temperature-treated microspores in cultivation medium with at least one live plant ovary to produce regenerative maize tissue, wherein the cultivation medium has an osmolarity between about 300 mOsm and about 500 mOsm and comprises kinetin, 2,4-dichlorophenoxyacetic acid, and phenylacetic acid; and
(e) regenerating maize plants from the regenerative maize tissue.

48. A method of producing regenerative maize tissue from maize microspores, comprising:
(a) selecting maize plant material comprising maize microspores at a developmental stage amenable to androgenic induction;
(b) incubating the microspores in incubation medium at a temperature and osmolarity effective to induce androgenesis to obtain temperature-treated microspores;
(c) isolating the temperature-treated microspores; and
(d) cultivating the isolated, temperature-treated microspores in cultivation medium with either at least one live plant ovary or ovary-conditioned medium to produce regenerative maize tissue, wherein the cultivation medium has an osmolarity between about 300 mOsm and about 500 mOsm and comprises at least one cytokinin and at least one auxin.
